(19) United States
(12) Patent Application Publication
(10) Pub. No.: US 2026/0299066 A1
(43) Pub. Date: Oct. 1, 2026

(54) RESTING-STATE FUNCTIONAL MAGNETIC RESONANCE IMAGING (MRI) DERIVED FROM DYNAMIC SUSCEPTIBILITY CONTRAST PERFUSION MRI

(71) Applicant: The Regents of the University of California, Oakland, CA (US)

(72) Inventors: Benjamin M. Ellingson, Redondo Beach, CA (US); Nicholas S. Cho, Glen Rock, NJ (US); Chencai Wang, Oakland, CA (US)

(21) Appl. No.: 19/632,747

(22) Filed: Mar. 30, 2026

Related U.S. Application Data
(60) Provisional application No. 63/780,042, filed on Mar. 28, 2025.

(51) Int. Cl. G01R 33/48; A61B 5/00; A61B 5/026; G01R 33/56

(52) U.S. Cl. CPC G01R 33/4806; A61B 5/0042; A61B 5/0263; A61B 5/7267; G01R 33/5601; G01R 33/5608; A61B 2576/026

(57) ABSTRACT

Systems and methods performing resting-state functional magnetic resonance imaging (fMRI) for a subject are provided that include receiving dynamic susceptibility contrast (DSC) perfusion magnetic resonance (MR) data for the subject, generating pseudo-resting-state fMRI data for the subject from the DSC perfusion MR data, generating one or more of an image map and a quantitative imaging measurement based on the pseudo-resting-state fMRI data, and generating a report comprising one or more of the image map and quantitative imaging measurement. The pseudo-rs-fMRI data can be generated from the DSC perfusion MR data by modeling a signal of contrast agent bolus on the DSC perfusion MR data and removing the signal of the contrast agent bolus from the DSC perfusion MR data.

### CROSS-REFERENCE TO RELATED APPLICATIONS

This application is based on, claims priority to, and incorporates herein by reference in its entirety U.S. Ser. No. 63/780,042 filed Mar. 28, 2025 and entitled “Resting-State Functional Magnetic Resonance Imaging (MRI) Derived From Dynamic Susceptibility Contrast (DSC) Perfusion MRI.”

### STATEMENT REGARDING FEDERALLY SPONSORED RESEARCH OR DEVELOPMENT

N/A

### BACKGROUND

In the United States, there are an estimated 700,000 patients who are currently diagnosed with a primary brain tumor. Although typical brain tumor management involves assessing tumor size from conventional, structural magnetic resonance imaging (MRI) techniques (e.g., T1-weighted post-contrast MRI), the utilization of advanced MRI techniques can provide valuable insights into tumor biology and brain function for improved clinical management.

Blood oxygenation level dependent (BOLD) functional MRI (fMRI), for example, can be used to assess changes in regional blood hemodynamics to assess brain activity. BOLD fMRI has an important role in neuro-oncological care for the pre-surgical mapping of eloquent cortex adjacent to brain tumors, particularly the language and motor networks. Pre-surgical fMRI mapping has been shown to reduce post-surgical morbidity for patients with brain tumors who undergo brain tumor resection compared to patients who did not undergo pre-surgical fMRI in a recent meta-analysis. Pre-surgical fMRI mapping is typically performed using task-based functional MRI (tb-fMRI) during which the patient carries out multiple paradigms in the scanner to generate activation maps of eloquent cortex (e.g., finger-tapping, sentence completion). However, tb-fMRI in the clinical setting may be challenging because image quality is reliant on optimal patient performance—which can be especially challenging for patients diagnosed with neurological conditions—as well as the availability of highly-trained personnel or advanced equipment to administer the paradigms.

Given these limitations, resting-state functional MRI (rs-fMRI) is emerging as a potential alternative to tb-fMRI in clinical settings. During an rs-fMRI scan, patients are scanned at “rest”, precluding the need for task paradigms. Image maps of resting-state networks can be generated by correlating the spontaneous fluctuations in BOLD signal of selected brain regions to the rest of the brain, which generates network maps of functional connectivity (FC) values consisting of functionally interconnected brain regions. Like tb-fMRI, rs-fMRI has also been shown to have clinical utility for pre-surgical mapping. Additionally, rs-fMRI is widely used in research settings to quantitatively assess brain alterations through resting-state network FC and to predict neurocognitive function and other clinical characteristics. The most well-known resting-state network is the default mode network, which includes key nodes in the medial prefrontal cortex & posterior cingulate cortex and is known for its associations with cognition and alterations in neurological conditions including Alzheimer's disease, stroke, and brain tumors.

Despite the potential usefulness of rs-fMRI, its clinical adoption for brain tumor management and other neurological diseases (e.g., stroke, multiple sclerosis, epilepsy, and Alzheimer's Disease) has remained limited to mostly advanced academic medical centers—and even then, only in limited circumstances—due to limitations of scan time, potentially resulting in this patient population being relatively understudied using rs-fMRI. There remains a need for the ability to perform resting-state analyses without additional scan time in clinical settings.

### SUMMARY

In accordance with an embodiment, a method for performing resting-state functional magnetic resonance imaging (fMRI) for a subject includes receiving, using a processor device, dynamic susceptibility contrast (DSC) perfusion magnetic resonance (MR) data for the subject, generating, using the processor device, pseudo-resting-state fMRI data for the subject from the DSC perfusion MR data, generating, using the processor device, one or more of an image map and a quantitative imaging measurement based on the pseudo-resting-state fMRI data, and generating, using the processor device, a report comprising one or more of the image map and quantitative imaging measurement.

In accordance with another embodiment, a system for performing resting-state functional magnetic resonance imaging (fMRI) for a subject, includes a memory that stores one or more computer readable media that includes instructions and one or more processor devices configured to execute the instructions of the computer readable media to receive dynamic susceptibility contrast (DSC) perfusion magnetic resonance (MR) data for the subject, generate pseudo-resting-state fMRI data for the subject from the DSC perfusion MR data, generate one or more of an image map and a quantitative imaging measurement based on the pseudo-resting-state fMRI data, and generate a report comprising one or more of the image map and quantitative imaging measurement.

### BRIEF DESCRIPTION OF THE DRAWINGS

The present invention will hereafter be described with reference to the accompanying drawings, wherein like reference numerals denote like elements.

FIG. 1 is a schematic diagram of an example system, in accordance with aspects of the present disclosure;

FIG. 2 illustrates a method for performing resting-state functional magnetic resonance imaging (rs-fMRI) using pseudo-rs-fMRI magnetic resonance (MR) data in accordance with an embodiment;

FIG. 3 illustrates an example method for generating pseudo-rs-fMRI data from dynamic susceptibility contrast (DSC) perfusion MR data in accordance with an embodiment;

FIG. 4 is a schematic diagram of an example magnetic resonance imaging (MRI) system in accordance with an embodiment; and

FIG. 5 is a block diagram of an example computer system in accordance with an embodiment.

### DETAILED DESCRIPTION

Dynamic susceptibility contrast (DSC) perfusion magnetic resonance imaging (MRI) is a standard of care MRI sequence for many patients with brain pathologies, including brain tumors, stroke, and neurodegenerative disorders. DSC perfusion MRI uses a gadolinium-based contrast agent administered to (e.g., injected into) the patient (or subject), and the brain is dynamically imaged to assess tumor vascularity (blood volume and blood flow) as the bolus passes through the brain's vasculature. DSC perfusion MRI is extensively used for the management of brain tumors such as for glioma subtyping and distinguishing tumor recurrence from treatment effects. For acute stroke, DSC perfusion MRI is routinely performed to determine the stroke penumbra (i.e., extent of damaged brain). DSC perfusion MRI can be acquired with, for example, a gradient-echo-based imaging sequence with similar T2*-weighting to a blood-oxygen-level-dependent (BOLD) resting-state functional MRI (rs-fMRI) acquisition.

As mentioned, a major barrier for the widespread use of clinical rs-fMRI is its, presently, requisite additional scan time, which is why the clinical implementation of rs-fMRI has generally been limited to very select institutions for pre-surgical planning. Notably, rs-fMRI is used in research settings to explore brain features to predict neurocognitive status and to study brain alterations associated with developmental disorders, aging, and disease, but it would be valuable to expand the availability of rs-fMRI to clinical settings to further study patient populations.

The present disclosure describes a system and method for resting-state functional magnetic resonance imaging (rs-fMRI) and analyses by using DSC perfusion MR data to generate pseudo-resting-state (pseudo-rs) fMRI data. In some embodiments, the pseudo-rs-fMRI data may be derived from the DSC perfusion MR data by modeling the signal of contrast agent bolus in the DSC perfusion MR data (e.g., the contrast agent bolus used in the acquisition of the DSC perfusion MR data) and removing the signal contribution of the contrast agent bolus from the DSC perfusion MR data. For example, in some embodiments, the signal of the contrast agent bolus can be removed from each voxel of the DSC perfusion MR data. In some embodiments, pseudo-rs-fMRI data may be derived without modeling the signal of the contrast agent bolus of the DSC perfusion MR data. For example, the pseudo-rs-fMRI data may be derived from the DSC perfusion MR data using signal cropping, signal augmentation, signal averaging, or artificial intelligence (e.g., a deep learning model). In some embodiments, pseudo-rs-fMRI data can be derived from one of the raw DSC perfusion MR data, leakage corrected DSC perfusion MR data, or other pre-processed or augmented (e.g., using artificial intelligence) DSC perfusion MR data, or synthetic (e.g., computationally modeled) DSC perfusion MR data. The generated pseudo-rs-fMRI data can be used to, for example, generate image maps of various resting-state networks (e.g., default mode network, motor network, language network, visual network, auditory network, attention network, executive network, a frontoparietal network, a salience network, a lesion-base network, or an associated inter-network connectivity) and perform functional connectivity analysis (e.g., to generate quantitative imaging (or image) measurements). Advantageously, in some embodiments, a single DSC perfusion MR acquisition (or scan) of a subject can be used to provide both perfusion MR metrics (e.g., relative cerebral blood volume (rCBV), relative cerebral blood flow (rCBF), percentage signal recovery (PSR), and mean transit time (MTT)) and resting-state fMRI metrics (e.g., network functional connectivity (FC), glioma-glioma FC, glioma-brain FC, and BOLD asynchrony).

The disclosed techniques utilizing pseudo-rs-fMRI data derived from DSC perfusion MR data may advantageously preclude the need for an additional rs-fMRI scan of a subject and provide the combined advantages of (i) network mapping in clinical settings when tb- or rs-fMRI is unavailable and (ii) expanding opportunities for additional rs-fMRI-related neuroscience research in subjects (e.g., a patient) with DSC perfusion MRI (iii) while still being able to perform brain perfusion analyses. In addition to using pseudo-rs-fMRI data for network maps and neurocognitive analyses, there are numerous other rs-fMRI analyses that have been employed for patients with brain tumors in recent years such as graph theory, within-tumor connectivity, and BOLD asynchrony that may now be more accessible using the disclosed pseudo-rs-fMRI-based approach. The applications of pseudo-rs-fMRI can be expanded to other patient populations beyond brain tumor that routinely undergo DSC perfusion MRI scanning as well, such as, for example, stroke, multiple sclerosis, epilepsy, and Alzheimer's Disease, amongst others. Furthermore, comprehensive neurocognitive assessments are challenging to implement in clinical settings. Non-invasive methods to predict cognitive impairment, such as through rs-fMRI, pseudo-rs-fMRI, or some other non-invasive imaging method, may be beneficial to perform when neuropsychological test batteries cannot be performed on patients.

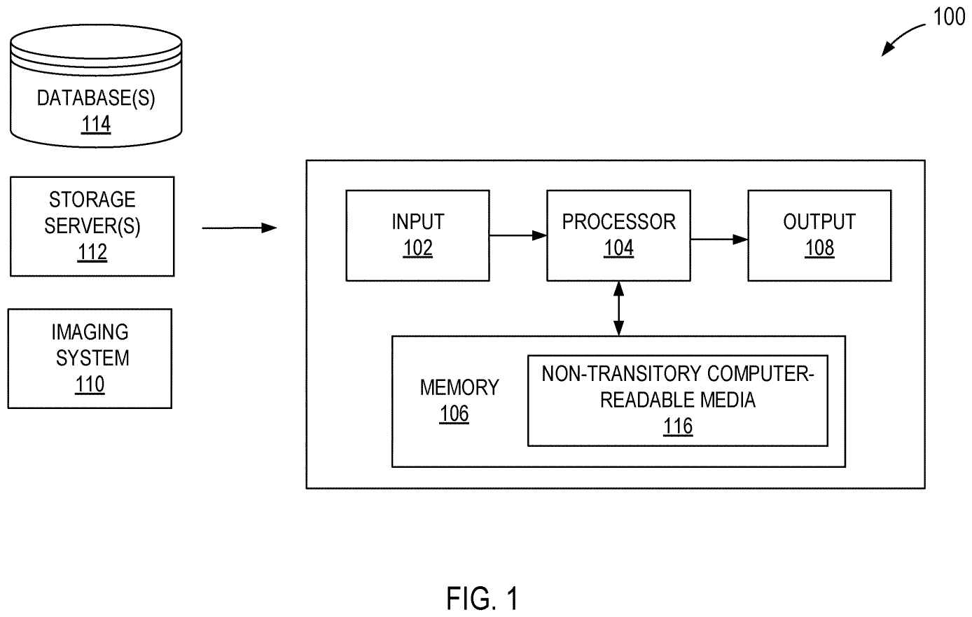

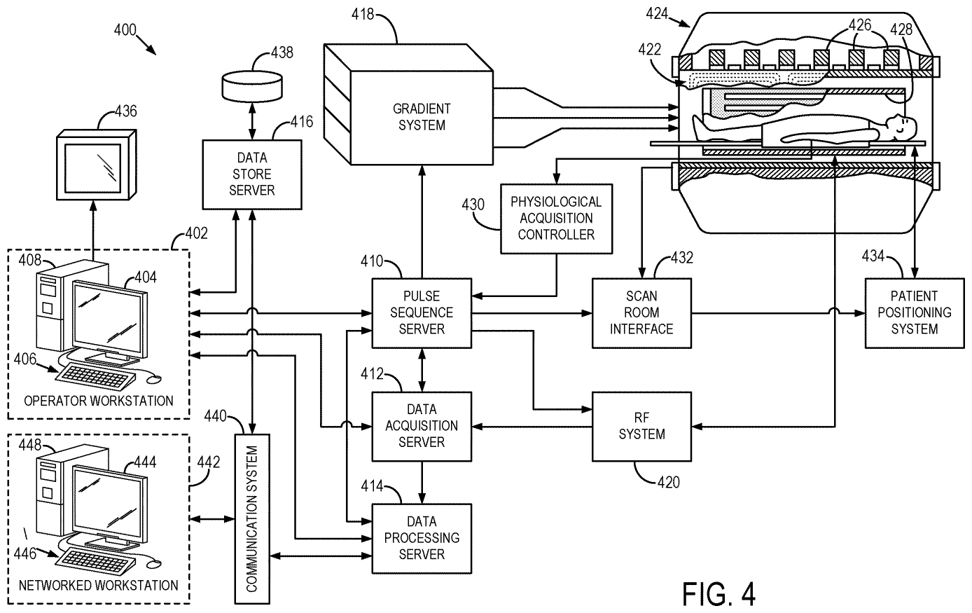

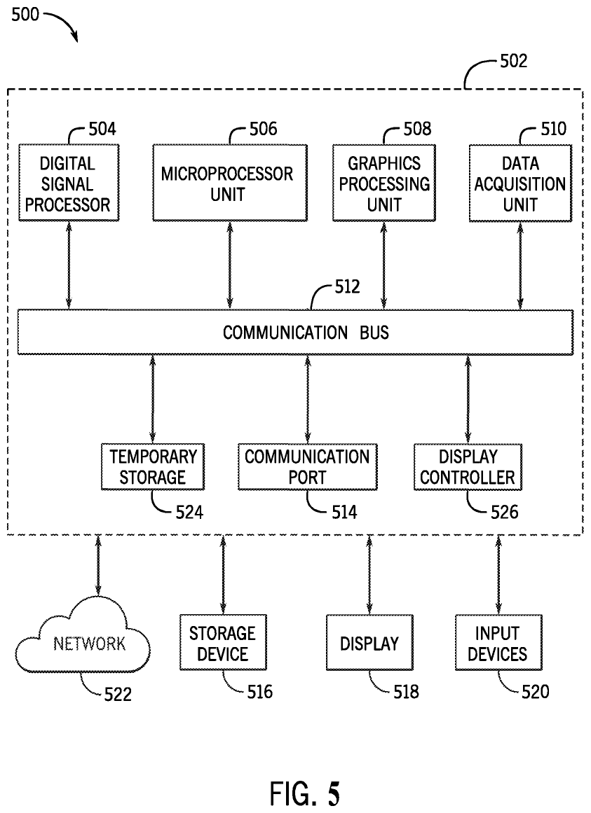

FIG. 1 is a schematic diagram of an example system, in accordance with aspects of the present disclosure. In general, the system 100 may include an input 102, a processor 104, a memory 106, and an output 108, and may be configured to carry out a process for generating pseudo-rs-fMRI data from DSC perfusion MR data and analyzing the pseudo-rs-fMRI data in accordance with aspects of the present disclosure. As shown in FIG. 1, the system 100 may communicate with one or more of imaging system 110, storage servers 112, or databases 114, by way of a wired or wireless connection. In general, the system 100 may be any device, apparatus, or system configured for carrying out instructions for, and may operate as part of, or in collaboration with, various computers, systems, devices, machines, mainframes, networks, or servers, for example, an example computer system 500 as described below with respect to FIG. 5. In some embodiments, the system 100 may be a portable or mobile device, such as a cellular phone or smartphone, laptop, tablet, and the like. In this regard, the system 100 may be a system that is designed to integrate a variety of software and hardware capabilities and functionalities, and may be capable of operating autonomously. In addition, although shown as separate from the imaging system 110, in some aspects, the system 100, or portions thereof, may be part of, or incorporated into, the imaging system 110, such as the example magnetic resonance imaging (MRI) system 400 described with reference to FIG. 4. The processor 104 may be implemented as one or more processors (or processor devices) such as, for example, a programmable processor or combination of programmable processors, such as central processing units (CPUs), graphic processing units (GPUs), and the like.

In some embodiments, the input 102 may include different input elements such as a mouse, keyboard, touchpad, touch screen, buttons, and the like, for receiving various selections and operational instructions from a user. The input 102 may also include various drives and receptacles, such as flash drives, USB drives, CD/DVD drives, and other computer-readable medium receptacles, for receiving various data and information. To this end, input 102 may also include various communication ports and modules, such as Ethernet, Bluetooth, or WiFi, for exchanging data and information with these, and other external computers, systems, devices, machines, mainframes, servers or networks.

In addition to being configured to carry out various steps for operating the system 100, the processor 104 may also be programmed to generate pseudo-rs-fMRI data from DSC perfusion MR data and analyze the pseudo-rs-fMRI data according to embodiments described herein. The processor 104 may be configured to execute instructions, stored in a non-transitory computer-readable media 116. Although the non-transitory computer-readable media 116 is shown in FIG. 1 as included in the memory 106, it may be appreciated that instructions executable by the processor 104 may be additionally or alternatively stored in another data storage location having non-transitory computer-readable media.

In some embodiments, the processor 104 may be configured to receive and process DSC perfusion MR data for a subject. In some embodiments, the processor 104 may access information and data, including the DSC perfusion MR data for a subject, stored in the imaging system 110, storage server(s) 112, database(s) 114, PACS, or other storage location. Accordingly, in some embodiments, the DSC perfusion MR data of the subject may be previously acquired from a subject and retrieved from data storage. In some embodiments, the processor 104 may direct acquisition of DSC perfusion MR data of a subject, for example, as part of a prospective image acquisition workflow using an MRI system (e.g., MRI system 400 as described with reference to FIG. 4). The DSC perfusion MR data can be acquired using known acquisition techniques for DSC perfusion MRI. In one example, the DSC perfusion MR data can be acquired using a gradient echo sequence with T2*-weighting. In another example, the DSC perfusion MR data can be acquired using a DSC perfusion MR protocol that has been optimized in scan acquisition length for conducting pseudo-rs-fMRI and perfusion analyses. In some embodiments, the DSC perfusion MR data can be acquired, for example, at various MR field strengths (e.g., 1.5T, 3T, 7T), with various acceleration schemes (e.g., undersampling, simultaneous multi-slice), and/or with a single echo or multi-echo technique. The DSC perfusion MR data can be a time series of data or images acquired at one or more time points or time periods. As mentioned, the DSC perfusion MR data can be acquired using a contrast agent (e.g., gadolinium-based contrast agent) administered (e.g., injected) into the subject. In some aspects, the DSC perfusion MR data can be acquired with or without a pre-load contrast agent bolus (e.g., a smaller dose of contrast agent administered before the main, larger contrast agent bolus). In some aspects, the DSC perfusion MR data can be acquired with a low-dose contrast agent or non-dosage of contrast agent.

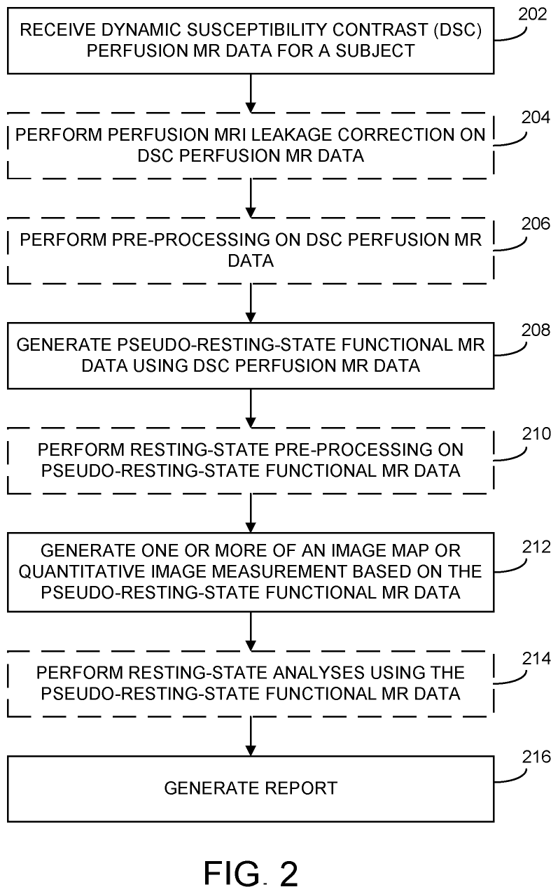

In some embodiments, the DSC perfusion MR data may be synthetic (e.g., computationally modeled) DSC perfusion MR data generated for the subject using, for example, computational modeling or artificial intelligence. The processor 104 may also preprocess the received DSC perfusion MR data. For example, the received DSC perfusion MR data (e.g., raw DSC perfusion MR data) can be pre-processed for signal filtering, signal cropping, signal attenuation, signal augmentation (e.g., signal padding or signal duplication), signal averaging, and motion correction. In some embodiments, the processor 104 may optionally process the received DSC perfusion MR data by performing leakage correction (e.g., using a bi-directional leakage correction technique as described below with respect to FIG. 2) to create leakage corrected DSC perfusion MR data (or signal). In some embodiments, the received DSC perfusion MR data of the subject can be pre-processed or augmented using, for example, artificial intelligence. The processor 104 can also be configured to determine one or more perfusion MR metrics (e.g., relative cerebral blood volume (rCBV), relative cerebral blood flow (rCBF), percentage signal recovery (PSR), and mean transit time (MTT)) from the DSC perfusion MR data.

The processor 104 may also be configured to generate or derive pseudo-rs-fMRI data from the DSC perfusion MR data for the subject (e.g., the raw DSC perfusion MR data, leakage corrected DSC perfusion MR data, other pre-processed or augmented (e.g., signal filtering, signal cropping, signal attenuation, and/or motion correction) DSC perfusion MR data, or synthetic DSC perfusion MR data). In some embodiments, the pseudo-rs-fMRI data can be generated (or extracted) by modeling (e.g., parametric modeling) the contrast agent bolus signal (or other signal contributions) from the DSC perfusion MR data (e.g., raw DSC perfusion MR data, leakage-corrected DSC perfusion MR data, simulated/synthetic DSC perfusion MR data, and/or any variation of pre-processed DSC perfusion MR data). Known contrast agent bolus modeling approaches can be used to model the contrast agent bolus signal. In one example, the contrast agent bolus can be modeled using a Gamma-variate fit model. In some embodiments, the contrast agent bolus signal may be modeled using voxel-wise, slice-wise, or regional-based modeling. The processor 104 can then remove the modeled contrast agent bolus signal from each voxel of the DSC perfusion MR data to create the pseudo-rs-fMRI data for the subject. Various techniques can be used by the processor 104 to remove the modeled contrast agent bolus signal from the DSC perfusion MR data. In one example, the modeled contrast agent bolus signal can be removed from the DSC perfusion MR data (or signal) by subtracting (e.g., voxel-wise) the modeled contrast agent bolus signal from the DSC perfusion MR data. In another example, the modeled contrast agent bolus signal can be removed from the DSC perfusion MR data (or signal) by filtering or deconvoluting (e.g., voxel-wise) the modeled contrast agent bolus signal from the DSC perfusion MR data. In yet another example, the modeled contrast agent bolus signal can be removed from the DSC perfusion MR data (or signal) by excluding or modifying image volumes or image volume time points involving signal of the contrast agent bolus (e.g., the volumes with high bolus effect) from the DSC perfusion MR data.

In some embodiments, the pseudo-rs-fMRI data can be generated (or extracted) by using signal cropping, for example, a set of pre-contrast agent bolus baseline timepoints acquired before administration of the contrast agent) or a set of post-bolus tail timepoints acquired after the first pass and recirculation effects of the contrast agent have subsided can be used as the pseudo-rs-fMRI data (e.g., a pseudo-rs-fMRI timeseries). In some embodiments, the pseudo-rs-fMRI data can be generated (or extracted) using signal augmentation or averaging. For example, pseudo-rs-fMRI data can be generated by averaging the pre-bolus and post-bolus data segments of the DSC perfusion MR data to increase the effective time series length, or pseudo-rs-fMRI data can be generated by duplicating or padding the bolus-free pre- and post-bolus data segments of the DSC perfusion MR data. In some embodiments, the pseudo-rs-fMRI data can be generated (or extracted) using artificial intelligence-based approaches in which a deep learning model (e.g., a convolution neural network (CNN), U-Net, or transformer architecture) can take the full DSC perfusion MR data time series as input and output pseudo-rs-fMRI data (or signal) with the bolus contribution removed. In one example, the deep learning model can be trained on paired DSC perfusion MR data and resting-state fMRI data to learn the separation without explicit parametric bolus modeling.

Once the pseudo-rs-fMRI data has been generated (or derived) from the DSC perfusion MR data, the processor 104 may then perform resting-state fMRI pre-processing techniques on the pseudo-rs-fMRI data such as, for example, registration, denoising, bandpass filtering, slice-timing correction, motion regression, functional realignment/unwarping, segmentation, outlier identification, etc. Based on the generated pseudo-s-fMRI data (either pre-processed or not pre-processed with rs-fMRI pre-processing techniques), the processor 104 can be configured to perform resting-state analyses including, for example, generating a map (e.g., an image map) of one or more resting-state networks (e.g., for pre-surgical planning), generating one or more quantitative imaging (or image) measurements, and performing quantitative functional connectivity analysis. The resting-state networks can include, for example, default mode network, motor network, language network, visual network, auditory network, attention network, executive network, a frontoparietal network, a salience network, a lesion-based network, or an associated inter-network connectivity. In some embodiments, resting-state network maps can be generated using the pseudo-rs-fMRI within standard atlas spaces (e.g., Montreal Neurological Institute (MNI) atlas) or within other spaces (e.g., native image space). In some embodiments, the quantitative imaging measurements can include, for example, functional connectivity strength (e.g., Pearson or partial correlation coefficients, Fisher z-transformed values), graph theory metrics (e.g., nodal degree, clustering coefficient, betweenness centrality, global and local efficiency, small-worldness, modularity), amplitude of low-frequency fluctuations (ALFF) and fractional ALFF, regional homogeneity (ReHo), BOLD asynchrony metrics, intralesional functional connectivity values, lesion-brain functional connectivity values, and network-level summary statistics such as within-network and between-network connectivity ratios. In some embodiments, the functional connectivity analysis performed using the pseudo-rs-fMRI can include assessing functional connectivity differences related to cognition or other clinical data. In some embodiments, the pseudo-rs-fMRI data can be assessed using one or more quantitative approaches (e.g., seed-to-voxel, ROI-ROI, and independent component analysis). In some embodiments, the pseudo-rs-fMRI data can be used for quantitative analyses using graph theory, intralesional connectivity, lesion-brain connectivity, and BOLD asynchrony. In some embodiments, the pseudo-rs-fMRI data can be used to create network maps and perform functional connectivity (FC) analyses related to cognition. For example, the pseudo-rs-fMRI can be used to estimate neurological or cognitive impairment (e.g., in a subject with a brain tumor) based on patterns in brain activity, or functional connectivity.

The processor 104 may also be configured to generate a report, in any form, and provide the report via output 108. In some aspects, the report may include various raw or processed maps or images, perfusion MR metrics (e.g., rCBV, rCBF, PSR, and MTT), and rs-fMRI analyses and information such as resting-state networks maps, quantitative imaging measurements and quantitative functional connectivity analysis.

FIG. 2 illustrates a method for performing resting-state functional magnetic resonance imaging (rs-fMRI) using pseudo-rs-fMRI magnetic resonance (MR) data in accordance with an embodiment. The process illustrated in FIG. 2 is described as being carried out by the system illustrated in FIG. 1, however, in some examples, the process of FIG. 2 may be implemented by another system. Although the blocks of the process are illustrated in a particular order, in some embodiments, one or more blocks may be executed in a different order than illustrated in FIG. 2, or may be bypassed. The process may be implemented by a processing system including at least one electronic processor, where the at least one electronic processor may be or include a processor as previously described (e.g., including one or more individual processor devices) or as described further below with respect to FIGS. 4 and 5.

At block 202, DSC perfusion MR data for a subject may be received, for example, as an input 102 to processor 104. In some embodiments, the DSC perfusion MR data for the subject can be stored in and retrieved from an imaging system 110 (e.g., MRI system 400 described below with respect to FIG. 4), storage server(s) 112, database(s) 114, PACS, or other storage location. In some embodiments, the DSC perfusion MR data of the subject may be previously acquired from a subject and retrieved from data storage. In some embodiments, the DSC perfusion MR data may be acquired in real time from the subject, for example, as part of a prospective image acquisition workflow using an MRI system (e.g., MRI system 400 as described with reference to FIG. 4). The DSC perfusion MR data can be acquired using known acquisition techniques for DSC perfusion MRI, for example, a gradient echo sequence with T2*-weighting, and can be acquired using a contrast agent (e.g., gadolinium-based contrast agent) administered (e.g., injected) into the subject. In some embodiments, the DSC perfusion MR data may be synthetic (e.g., computationally modeled) DSC perfusion MR data generated for the subject using, for example, computational modeling or artificial intelligence. In some embodiments, the DSC perfusion MR data can be a time series of data or images acquired at one or more time points or time periods.

In some embodiments, the DSC perfusion MR data can be acquired using a DSC perfusion MR protocol that has been optimized in scan acquisition length for conducting pseudo-rs-fMRI and perfusion analyses. In one example, the scan acquisition length of the DSC perfusion MRI acquisition can be increased to be compliant with rs-FMRI guidelines and limited within a suggested maximal delay between contrast agent injection and data acquisition for a standardized brain imaging protocol, and the DSC perfusion MR signal can be cropped to a shorter duration to be compliant with DSC perfusion MR I guidelines for, for example, brain tumors.

At block 204, leakage correction may optionally be performed on the DSC perfusion MR data, for example, using processor 104, to generate leakage corrected DSC perfusion MR data. In some embodiments, the leakage correction of the DSC perfusion MR data can be performed using known leakage correction techniques. In one example, the leakage correction may be a bidirectional leakage correction technique as described in U.S. Pat. No. 10,973,433, issued Apr. 13, 2021, hereinafter incorporated by reference in its entirety. The example bidirectional leakage correction technique, herein also referred to as a bidirectional model, can account for bidirectional contrast agent exchange and transport between intra- and extravascular spaces. In the example bidirectional model, DSC perfusion MR data, for example, a dynamic series of T2*-weighted MR images, can be transformed into an image depicting a more accurate estimate of relative blood volume, particularly during pathological circumstances when contrast agent has leaked from the blood vessels. At block 206, the received DSC perfusion MR data (e.g., raw DSC perfusion ME data) may also optionally be pre-processed using processor 104, for example, using one or more of signal filtering, signal cropping, signal attenuation, signal augmentation, and motion correction. In some embodiments, the received DSC perfusion MR data of the subject can be pre-processed or augmented using, for example, artificial intelligence.

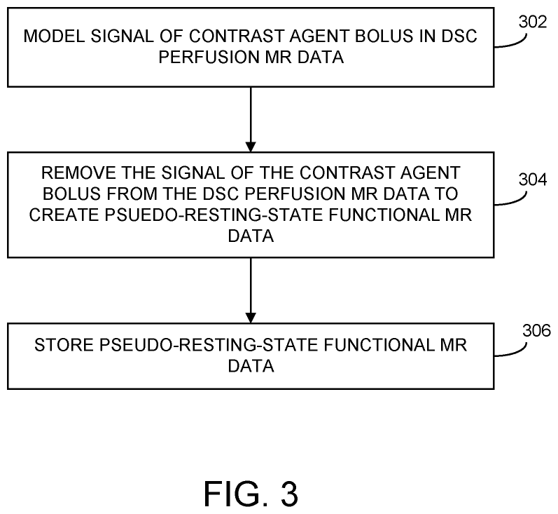

At block 208, pseudo-rs-FMRI data may be generated or derived from the DSC perfusion MR data (e.g., the raw DSC perfusion MR data, leakage corrected DSC perfusion MR data, other pre-processed or augmented DSC perfusion MR data, or synthetic DSC perfusion MR data), for example, using the processor 104. As mentioned, in some embodiments, the pseudo-rs-fMRI data can be generated by modeling the contrast agent bolus signal from the DSC perfusion MR data and removing the modeled contrast agent bolus signal from the DSC perfusion MR data. FIG. 3 illustrates an example method for generating pseudo-rs-fMRI data from dynamic susceptibility contrast (DSC) perfusion MR data in accordance with an embodiment. The process illustrated in FIG. 3 is described as being carried out by the system illustrated in FIG. 1, however, in some examples, the process of FIG. 3 may be implemented by another system. Although the blocks of the process are illustrated in a particular order, in some embodiments, one or more blocks may be executed in a different order than illustrated in FIG. 3, or may be bypassed. The process may be implemented by a processing system including at least one electronic processor, where the at least one electronic processor may be or include a processor as previously described (e.g., including one or more individual processor devices) or as described further below with respect to FIGS. 4 and 5.

Referring to FIG. 3, at block 302, a contrast agent bolus signal (or other signal contributions) in the DSC perfusion MR data can be modeled using a contrast agent modeling approach such as, for example a Gamma-variate fit model. In some embodiments, the contrast agent bolus signal may be modeled using voxel-wise, slice-wise, or regional-based modeling. At block 304, the modeled contrast agent bolus signal can be removed (e.g., using the processor 104) from the from each voxel of the of the DSC perfusion MR data to create the pseudo-rs-fMRI data, for example a residual pseudo-rs-fMRI signal. In one example, the modeled contrast agent bolus signal can be removed from the DSC perfusion MR data (or signal) by subtracting (e.g., voxel-wise) the modeled contrast agent bolus signal from the DSC perfusion MR data. In another example, the modeled contrast agent bolus signal can be removed from the DSC perfusion MR data (or signal) by filtering or deconvoluting (e.g., voxel-wise) the modeled contrast agent bolus signal from the DSC perfusion MR data. In yet another example, the modeled contrast agent bolus signal can be removed from the DSC perfusion MR data (or signal) by excluding or modifying image volumes or image volume time points involving signal of the contrast agent bolus (e.g., the volumes with high bolus effect) from the DSC perfusion MR data. At block 306, in some embodiments, the generated pseudo-rs-fMRI data can be stored in data storage, for example, data storage of an imaging system 110 (e.g., disc storage 638 of the example MRI system 400 shown in FIG. 4), storage server(s) 112, database(s) 114, or storage device 516 of the example computer system 500 shown in FIG. 5.

Referring again to FIG. 2, at block 208 various other techniques can be used to generate the pseudo-rs-FMRI data from the DSC perfusion MR data. As mentioned, in some embodiments, the pseudo-rs-fMRI data can be generated (or extracted) by using signal cropping, for example, a set of pre-contrast agent bolus baseline timepoints acquired before administration of the contrast agent) or a set of post-bolus tail timepoints acquired after the first pass and recirculation effects of the contrast agent have subsided can be used as the pseudo-rs-fMRI data (e.g., a pseudo-rs-fMRI timeseries). In some embodiments, the pseudo-rs-fMRI data can be generated (or extracted) using signal augmentation or averaging. For example, pseudo-rs-fMRI data can be generated by averaging the pre-bolus and post-bolus data segments of the DSC perfusion MR data to increase the effective time series length, or pseudo-rs-fMRI data can be generated by duplicating or padding the bolus-free pre- and post-bolus data segments of the DSC perfusion MR data. In some embodiments, the pseudo-rs-fMRI data can be generated (or extracted) using artificial intelligence-based approaches in which a deep learning model (e.g., a convolution neural network (CNN), U-Net, or transformer architecture) can take the full DSC perfusion MR data time series as input and output pseudo-rs-fMRI data (or signal) with the bolus contribution removed. In one example, the deep learning model can be trained on paired DSC perfusion MR data and resting-state fMRI data to learn the separation without explicit parametric bolus modeling.

At block 210, resting-state pre-processing may optionally be performed (e.g., using processor 104) on the generated pseudo-rs-fMRI data. The resting-state pre-processing techniques can include one or more of, for example, registration, denoising, bandpass filtering, slice-timing correction, motion regression, functional realignment/unwarping, segmentation, outlier identification, etc. In one example, the pseudo-rs-FMRI data can be registered to standard atlas spaces (e.g., the Montreal Neurological Institute (MNI) atlas), or within other spaces (e.g., native image space).

At block 212, one or more of an image map or quantitative image measurement(s) may be generated (e.g., using processor 104) based on the pseudo-rs-fMRI data (either pre-processed or not pre-processed with rs-fMRI pre-processing techniques) generated at block 208, for example, generating a map (e.g., an image map) of one or more resting-state networks, generating one or more quantitative imaging measurements, and performing quantitative functional connectivity analysis. In some embodiments, one or more resting-state networks can be mapped (e.g., an image map), for example, default mode network, motor network, language network, visual network, auditory network, attention network, executive network, a frontoparietal network, a salience network, a lesion-base network, or an associated inter-network connectivity. In some embodiments, the resting-state network maps can be generated using the pseudo-rs-fMRI data within standard atlas spaces (e.g., Montreal Neurological Institute (MNI) atlas) or within other spaces (e.g., native image space). Known methods can be used to generate the map(s) of resting-state networks using the pseudo-rs-fMRI data. In one example, seed-to-voxel maps can be generated using consistent seed regions of interest in the MNI atlas space. In this example, the default mode network can be generated by seeding the medial prefrontal cortex region of interest (a node of the default mode network), the motor network can be generated by seeding the left lateral sensorimotor cortex region of interest (a node of the motor network), and the language network can be generated by seeding the left inferior frontal gyrus region of interest (a node of the language network).

In some embodiments, the quantitative imaging measurements can include, for example, functional connectivity strength (e.g., Pearson or partial correlation coefficients, Fisher z-transformed values), graph theory metrics (e.g., nodal degree, clustering coefficient, betweenness centrality, global and local efficiency, small-worldness, modularity), amplitude of low-frequency fluctuations (ALFF) and fractional ALFF, regional homogeneity (ReHo), BOLD asynchrony metrics, intralesional functional connectivity values, lesion-brain functional connectivity values, and network-level summary statistics such as within-network and between-network connectivity ratios. In some embodiments, the functional connectivity analysis performed using the pseudo-rs-fMRI can include assessing functional connectivity differences related to cognition or other clinical data. In some embodiments, the pseudo-rs-fMRI data can be assessed using one or more quantitative approaches (e.g., seed-to-voxel, ROI-ROI, and independent component analysis). In some embodiments, the pseudo-rs-fMRI data can be used for quantitative analyses using graph theory. In some embodiments, the generated quantitative imaging measurement can be utilized to assess, for example, lesion biology, brain characteristics, clinical data, or neurocognitive function. In some embodiments, the pseudo-rs-fMRI data can be used to determine a prediction of neurocognitive function.

At block 214, additional resting-state analyses can optionally be performed (e.g., using processor 104) using the pseudo-rs-fMRI data, for example, quantitative functional connectivity (FC) analysis. The quantitative functional connectivity analysis can include, for example, network functional connectivity, glioma-glioma functional connectivity (or intralesional connectivity), glioma-brain functional connectivity (or lesion-brain connectivity), and blood-oxygen-level-dependence (BOLD) asynchrony. In some embodiments, the pseudo-rs-fMRI data can be used to perform functional connectivity analysis related to cognition. For example, the pseudo-rs-fMRI can be used to estimate neurological or cognitive impairment (e.g., in a subject with a brain tumor) based on patterns in brain activity, or functional connectivity. In some embodiments, analyses can be performed using the pseudo-rs-fMRI data and a set of clinical data.

At block 216, a report having the one or more image map or quantitative imaging measurement can be generated, for example, using processor 104. The report can include, for example, raw or processed maps or images, and rs-fMRI analyses and information such as resting-state networks maps, quantitative imaging measurements and quantitative functional connectivity analysis. As mentioned, in some embodiments, the processor 104 can be configured to generate perfusion MR metrics (e.g., rCBV, rCBF, PSR, and MTT) using the DSC perfusion MR data of the subject. The perfusion MR metrics may also be included in a report generated by the processor 104. In some embodiments, the generated report can be stored in data storage, for example, data storage of an imaging system 110 (e.g., disc storage 638 of the example MRI system 400 shown in FIG. 4), storage server(s) 112, database(s) 114, or storage device 516 of the example computer system 500 shown in FIG. 5.

FIG. 4 is a block diagram of an example magnetic resonance imaging (MRI) system in accordance with an embodiment. MRI system. In some embodiments, MRI system 400 may be used to implement various systems and methods described herein. In some embodiments, the disclosed systems and methods may be designed to accompany the MRI system 400. MRI system 400 includes an operator workstation 402, which may include a display 404, one or more input devices 406 (e.g., a keyboard, a mouse), and a processor 408. The processor 408 may include a commercially available programmable machine running a commercially available operating system. The operator workstation 402 provides an operator interface that facilitates entering scan parameters into the MRI system 400. The operator workstation 402 may be coupled to different servers, including, for example, a pulse sequence server 410, a data acquisition server 412, a data processing server 414, and a data store server 416. The operator workstation 402 and the servers 410, 412, 414, and 416 may be connected via a communication system 440, which may include any suitable network connection, whether wired, wireless, or a combination of both.

The pulse sequence server 410 functions in response to instructions provided by the operator workstation 402 to operate a gradient system 418 and a radiofrequency (“RF”) system 420. Gradient waveforms for performing a prescribed scan are produced and applied to the gradient system 418, which then excites gradient coils in an assembly 422 to produce the magnetic field gradients Gₓ, G_{y}, and G_{z} that are used for spatially encoding magnetic resonance signals. The gradient coil assembly 422 forms part of a magnet assembly 424 that includes a polarizing magnet 426 and a whole-body RF coil 428 and/or a local coil (not shown).

RF waveforms are applied by the RF system 420 to the RF coil 428, or a separate local coil to perform the prescribed magnetic resonance pulse sequence. Responsive magnetic resonance signals detected by the RF coil 428, or a separate local coil, are received by the RF system 420. The responsive magnetic resonance signals may be amplified, demodulated, filtered, and digitized under direction of commands produced by the pulse sequence server 410. The RF system 420 includes an RF transmitter for producing a wide variety of RF pulses used in MRI pulse sequences. The RF transmitter is responsive to the prescribed scan and direction from the pulse sequence server 410 to produce RF pulses of the desired frequency, phase, and pulse amplitude waveform. The generated RF pulses may be applied to the whole-body RF coil 428 or to one or more local coils or coil arrays.

The RF system 420 also includes one or more RF receiver channels. An RF receiver channel includes an RF preamplifier that amplifies the magnetic resonance signal received by the coil 428 to which it is connected, and a detector that detects and digitizes the I and Q quadrature components of the received magnetic resonance signal. The magnitude of the received magnetic resonance signal may, therefore, be determined at a sampled point by the square root of the sum of the squares of the I and Q components:

$$M = \sqrt{{I}^{2} + {Q}^{2}} \qquad ( 1 )$$

and the phase of the received magnetic resonance signal may also be determined according to the following relationship:

$$φ = {tan}^{- 1} ( \frac{Q}{I} ) \qquad ( 2 )$$

The pulse sequence server 410 may receive patient data from a physiological acquisition controller 430. By way of example, the physiological acquisition controller 430 may receive signals from a number of different sensors connected to the patient, including electrocardiograph (“ECG”) signals from electrodes, or respiratory signals from a respiratory bellows or other respiratory monitoring devices. These signals may be used by the pulse sequence server 410 to synchronize, or “gate,” the performance of the scan with the subject's heartbeat or respiration.

The pulse sequence server 410 may also connect to a scan room interface circuit 432 that receives signals from various sensors associated with the condition of the patient and the magnet system. Through the scan room interface circuit 432, a patient positioning system 434 can receive commands to move the patient to desired positions during the scan.

The digitized magnetic resonance signal samples produced by the RF system 420 are received by the data acquisition server 412. The data acquisition server 412 operates in response to instructions downloaded from the operator workstation 102 to receive the real-time magnetic resonance data and provide buffer storage, so that data is not lost by data overrun. In some scans, the data acquisition server 412 passes the acquired magnetic resonance data to the data processor server 414. In scans that require information derived from acquired magnetic resonance data to control the further performance of the scan, the data acquisition server 412 may be programmed to produce such information and convey it to the pulse sequence server 410. For example, during pre-scans, magnetic resonance data may be acquired and used to calibrate the pulse sequence performed by the pulse sequence server 410. As another example, navigator signals may be acquired and used to adjust the operating parameters of the RF system 420 or the gradient system 418, or to control the view order in which k-space is sampled. In still another example, the data acquisition server 412 may also process magnetic resonance signals used to detect the arrival of a contrast agent in a DSC perfusion MRI scan. For example, the data acquisition server 412 may acquire magnetic resonance data and processes it in real-time to produce information that is used to control the scan.

The data processing server 414 receives magnetic resonance data from the data acquisition server 412 and processes the magnetic resonance data in accordance with instructions provided by the operator workstation 402. Such processing may include, for example, reconstructing two-dimensional or three-dimensional images by performing a Fourier transformation of raw k-space data, performing other image reconstruction algorithms (e.g., iterative or back-projection reconstruction algorithms), applying filters to raw k-space data or to reconstructed images, generating functional magnetic resonance images, or calculating motion or flow images.

Images reconstructed by the data processing server 414 are conveyed back to the operator workstation 402 for storage. Real-time images may be stored in a data base memory cache, from which they may be output to operator display 404 or a display 436. Batch mode images or selected real time images may be stored in a host database on disc storage 438. When such images have been reconstructed and transferred to storage, the data processing server 414 may notify the data store server 416 on the operator workstation 402. The operator workstation 402 may be used by an operator to archive the images, produce films, or send the images via a network to other facilities.

The MRI system 400 may also include one or more networked workstations 442. For example, a networked workstation 442 may include a display 444, one or more input devices 446 (e.g., a keyboard, a mouse), and a processor 448. The networked workstation 442 may be located within the same facility as the operator workstation 402, or in a different facility, such as a different healthcare institution or clinic.

The networked workstation 442 may gain remote access to the data processing server 414 or data store server 416 via the communication system 440. Accordingly, multiple networked workstations 442 may have access to the data processing server 414 and the data store server 416. In this manner, magnetic resonance data, reconstructed images, or other data may be exchanged between the data processing server 414 or the data store server 416 and the networked workstations 442, such that the data or images may be remotely processed by a networked workstation 442.

FIG. 5 is a block diagram of an example computer system in accordance with an embodiment. Computer system 500 may be used to implement the systems and methods described herein. In some embodiments, the computer system 500 may be a workstation, a notebook computer, a tablet device, a mobile device, a multimedia device, a network server, a mainframe, one or more controllers, one or more microcontrollers, or any other general-purpose or application-specific computing device. The computer system 500 may operate autonomously or semi-autonomously, or may read executable software instructions from the memory or storage device 516 or a computer-readable medium (e.g., a hard drive, a CD-ROM, flash memory), or may receive instructions via the input device 520 from a user, or any other source logically connected to a computer or device, such as another networked computer or server. Thus, in some embodiments, the computer system 500 can also include any suitable device for reading computer-readable storage media.

Data, such as data acquired with, for example, an imaging system (e.g., a magnetic resonance imaging (MRI) system, etc.), may be provided to the computer system 500 from a data storage device 516, and these data are received in a processing unit 502. In some embodiments, the processing unit 502 included one or more processors. For example, the processing unit 502 may include one or more of a digital signal processor (DSP) 504, a microprocessor unit (MPU) 506, and a graphic processing unit (GPU) 508. The processing unit 502 also includes a data acquisition unit 510 that is configured to electronically receive data to be processed. The DSP 504, MPU 506, GPU 508, and data acquisition unit 510 are all coupled to a communication bus 512. The communication bus 512 may be, for example, a group of wires, or a hardware used for switching data between the peripherals or between any component in the processing unit 502.

The processing unit 502 may also include a communication port 514 in electronic communication with other devices, which may include a storage device 516, a display 518, and one or more input devices 520. Examples of an input device 520 include, but are not limited to, a keyboard, a mouse, and a touch screen through which a user can provide an input. The storage device 516 may be configured to store data, which may include data such as, for example, DSC perfusion MRI data, pseudo-rs-fMRI data, network maps, functional connectivity data, etc., whether these data are provided to, or processed by, the processing unit 502. The display 518 may be used to display images and other information, such as patient health data, and so on.

The processing unit 502 can also be in electronic communication with a network 522 to transmit and receive data and other information. The communication port 514 can also be coupled to the processing unit 502 through a switched central resource, for example the communication bus 512. The processing unit 502 can also include temporary storage 524 and a display controller 526. The temporary storage 524 is configured to store temporary information. For example, the temporary storage can be a random access memory.

Computer-executable instructions for performing resting-state functional magnetic resonance imaging (rs-fMRI) using pseudo-rs-fMRI data derived from DSC perfusion MRI data according to the above-described methods may be stored on a form of computer readable media. Computer readable media includes volatile and nonvolatile, removable, and non-removable media implemented in any method or technology for storage of information such as computer readable instructions, data structures, program modules or other data. Computer readable media includes, but is not limited to, random access memory (RAM), read-only memory (ROM), electrically erasable programmable ROM (EEPROM), flash memory or other memory technology, compact disk ROM (CD-ROM), digital volatile disks (DVD) or other optical storage, magnetic cassettes, magnetic tape, magnetic disk storage or other magnetic storage devices, or any other medium which can be used to store the desired instructions and which may be accessed by a system (e.g., a computer), including by internet or other computer network form of access.

The present disclosure has been described in terms of one or more preferred embodiments, and it should be appreciated that many equivalents, alternatives, variations, and modifications, aside from those expressly stated, are possible and within the scope of the invention.

1. A method for performing resting-state functional magnetic resonance imaging (fMRI) for a subject, the method comprising:
receiving, using a processor device, dynamic susceptibility contrast (DSC) perfusion magnetic resonance (MR) data for the subject;
generating, using the processor device, pseudo-resting-state fMRI data for the subject from the DSC perfusion MR data;
generating, using the processor device, one or more of an image map and a quantitative imaging measurement based on the pseudo-resting-state fMRI data; and
generating, using the processor device, a report comprising one or more of the image map and quantitative imaging measurement.

2. The method according to claim 1, wherein generating, using the processor device, pseudo-resting-state fMRI data for the subject from the DSC perfusion MR data comprises:
modeling a signal of a contrast bolus in the DSC perfusion MR data; and
removing the signal of the contrast agent bolus from the DSC perfusion MR data.

3. The method according to claim 2, wherein removing the signal of the contrast agent bolus from the DSC perfusion MR data comprises subtracting the signal of the contrast agent bolus from the DSC perfusion MR data.

4. The method according to claim 2, wherein removing the signal of the contrast agent bolus from the DSC perfusion MR data comprises filtering out the signal of the contrast agent bolus from the DSC perfusion MR data.

5. The method according to claim 2, wherein removing the signal of the contrast agent bolus from the DSC perfusion MR data comprises removing image volume time points involving signal of the contrast agent bolus from the DSC perfusion MR data.

6. The method according to claim 1, further comprising performing, using the processor device, leakage correction on the DSC perfusion MR data.

7. The method according to claim 1, wherein generating, using the processor device, pseudo-resting-state fMRI data for the subject from the DSC perfusion MR data comprises performing one of signal cropping, signal augmentation, signal averaging, or processing using a deep learning model.

8. The method according to claim 7, further comprising performing, using the processor device, on the DSC perfusion MR data one of signal filtering, signal cropping, signal attenuation, signal augmentation, or motion correction.

9. The method according to claim 1, wherein the received DSC perfusion MR data is acquired with an acceleration technique.

10. The method according to claim 1, wherein the DSC perfusion MR data is synthetic DSC perfusion MR data.

11. The method according to claim 1, further comprising generating, using the processor device, functional connectivity data based on the pseudo-resting-state fMRI data.

12. The method according to claim 1, wherein the generated image map is an image map of a resting state network.

13. A system for performing resting-state functional magnetic resonance imaging (fMRI) for a subject, the system comprising:
a memory that stores one or more computer readable media that includes instructions; and
one or more processor devices configured to execute the instructions of the computer readable media to:
receive dynamic susceptibility contrast (DSC) perfusion magnetic resonance (MR) data for the subject;
generate pseudo-resting-state fMRI data for the subject from the DSC perfusion MR data;
generate one or more of an image map and a quantitative imaging measurement based on the pseudo-resting-state fMRI data; and
generate a report comprising one or more of the image map and quantitative imaging measurement.

14. The system according to claim 13, wherein the one or more processor devices are configured to execute the instructions of the computer readable media further to:
generate the pseudo-resting-state fMRI data from the DSC perfusion MR data by modeling a signal of a contrast bolus in the DSC perfusion MR data, and removing the signal of the contrast agent bolus from the DSC perfusion MR data.

15. The system according to claim 14, wherein the one or more processor devices are configured to execute the instructions of the computer readable media further to remove the signal of the contrast agent bolus from the DSC perfusion MR data by subtracting the signal of the contrast agent bolus from the DSC perfusion MR data.

16. The system according to claim 14, wherein the one or more processor devices are configured to execute the instructions of the computer readable media further to remove the signal of the contrast agent bolus from the DSC perfusion MR data by filtering out the signal of the contrast agent bolus from the DSC perfusion MR data.

17. The system according to claim 14, wherein the one or more processor devices are configured to execute the instructions of the computer readable media further to remove the signal of the contrast agent bolus from the DSC perfusion MR data by removing image volume time points involving signal of the contrast agent bolus from the DSC perfusion MR data.

18. The system according to claim 13, wherein the one or more processor devices are configured to execute the instructions of the computer readable media further to perform leakage correction on the DSC perfusion MR data.

19. The system according to claim 13, wherein the one or more processor devices are configured to execute the instructions of the computer readable media further to generate functional connectivity data based on the pseudo-resting-state fMRI data.

20. The system according to claim 13, wherein the one or more processor devices are configured to execute the instructions of the computer readable media further to generate pseudo-resting-state fMRI data for the subject from the DSC perfusion MR data by performing one of signal cropping, signal augmentation, signal averaging, or processing using a deep learning model.
